(19) United States
(12) Patent Application Publication
(10) Pub. No.: US 2024/0027410 A1
(43) Pub. Date: Jan. 25, 2024

(54) REACTOR ASSEMBLY FOR AN INDUSTRIAL WATER ANALYSIS DEVICE

(71) Applicant: HORIBA TOCADERO GMBH, Berlin (DE)

(72) Inventors: Oliver ROTHE, Steinhagen (DE); Rafael JECHOREK, Berlin (DE); Frank BEHRENDT, Berlin (DE); Martin GLITTENBERG, Wuppertal (DE)

(21) Appl. No.: 18/043,101

(22) PCT Filed: Aug. 28, 2020
(86) PCT No.: PCT/EP2020/074145

§ 371 Date: Feb. 27, 2023

(51) Int. Cl. G01N 31/12; G01N 33/18

(52) U.S. Cl. CPC G01N 31/12; G01N 33/1846

(57) ABSTRACT

The invention relates to a reactor assembly (1) for an industrial water analysis device (2), particularly using a high temperature oxidation method, the reactor assembly (1) comprises an outer reactor tube (6) and an inner reactor tube (8), the inner reactor tube (8) being removably inserted into the outer reactor tube (8). The invention further relates to an industrial water analysis device (2) comprising a housing (4) and the aforementioned reactor assembly (1), wherein the outer reactor tube (6) is fixedly mounted to the housing (4). Moreover, the invention relates to an inner reactor tube (8) for the reactor assembly (1).

The present invention relates to a reactor assembly for an industrial water analysis device such as a total organic carbon analyser, particularly using a high temperature oxidation method.

The total organic carbon (TOC) is one of the most important parameters in water analysis. It represents the total amount of carbon found in an organic compound and may thus allow drawing conclusions on the organic content of the water samples such as drinking water, raw water, groundwater, surface water, seawater, waste water, leachates or high-purity water. The organic compounds may come from natural organic matter, such as humic acids, fulvic acids, amines or urea, as well as synthetic sources, for instance detergents, pesticides, industrial chemicals.

During the total organic carbon analysis the sample, has to be oxidised. For this, the sample is introduced into a reactor tube in which it is oxidised under high heat to form carbon dioxide, which flows from the reactor tube into a detection unit, particularly using a non-dispersive infrared sensor (NDIR sensor) for detecting the carbon dioxide.

Industrial water analysis devices are configured for industrial use, i.e. for use 24/7 to continuously monitor specific content in water, such as TOC or TN_{b} (total nitrogen bound). For this, the industrial water analysis device may be installed on-line or even in-line and may be configured to automatically draw and analyse water samples according to a predetermined schedule. This distinguishes the industrial water analysis device from a water analysis device for laboratory use, which is employed only sporadically.

To do maintenance work on the industrial water analysis device, at least in some cases, both the industrial water analysis device and the system it is installed in need to be shut down. Frequent and long downtimes of the system lead to high costs.

Hence, there is a need for an industrial water analysis device or a reactor assembly for such an industrial water analysis device that is easily and quickly maintained.

This objective is achieved by providing a reactor assembly for an industrial water analysis device, the reactor assembly comprising an outer reactor tube and an inner reactor tube being removably inserted into the outer reactor tube.

The above-mentioned solution is advantageous since a single reactor tube of a common industrial water analysis device is replaced by a combination of an outer reactor tube and an inner reactor tube. The outer reactor tube may be fixed to the analyser in the same manner a single reactor tube would be fixed. However, the sample is introduced into the inner reactor tube, in which oxidation takes place and which is removably inserted into the outer reactor tube. Hence, a quick exchange of the inner reactor tube is possible. A tedious dismantling of the analyser to allow dismounting of the outer reactor tube or single reactor tube respectively, is not necessary.

As described in more detail on the following pages, the reactor assembly may in particular be configured for an industrial water analysis device using a high temperature oxidation method. In this case, the reactor assembly is particularly of advantage, as it is not necessary to switch off the heating unit during the exchange of the inner reactor tubes, preventing additional cooling off and/or heating up periods of the heating unit. Hence, long downtimes of the industrial water analysis device as well as the system it is installed in may be avoided due to maintenance of the reactor assembly.

The invention can be further improved by the following features, which are independent from one another with respect to their technical effects and which can be combined arbitrarily.

For example, the inner reactor tube and/or the outer reactor tube may comprise a cylindrical body extending along a longitudinal axis. In an assembled state, the outer reactor tube and the inner reactor tube may preferably be arranged coaxially to one another. With such a configuration, a uniform heat transfer between the outer and inner reactor tube is ensured.

The inner reactor tube may at least partially rest on the outer reactor tube, thus, preventing the inner reactor tube from slipping through the outer reactor tube. The inner reactor tube may preferably rest on the outer reactor tube along an insertion direction, in which the inner reactor tube is inserted into the outer reactor tube. This may be particularly advantageous if the insertion direction is essentially parallel to the gravitational force vector, so that the inner reactor tube may be held by the outer reactor tube via the gravitational force and no further measurements have to be taken for holding the inner reactor tube in the outer reactor tube.

This may, for example, be realised if the inner reactor tube comprises a radially outwards protruding collar having a radially extending abutment surface for resting on a complementary abutment surface of the outer reactor tube. Preferably, an inner wall of the outer reactor tube may comprise a radial recess, in which the collar may be received. The collar may particularly be fittingly received in the recess, at least in the radial direction, so that the inner reactor tube is automatically aligned and/or centred with respect to the outer reactor tube.

The inner reactor tube may comprise an inlet opening arranged at a first end of the inner reactor tube for receiving the sample and an outlet opening arranged at a second end of the inner reactor tube opposite the first end. When inserting the inner reactor tube into the outer reactor tube along the insertion direction, the second end may first enter the outer reactor tube.

In a particularly advantageous embodiment, the radially protruding collar may be positioned at the first end of the inner reactor tube. In this embodiment, the collar may also act as a flange increasing a surface area of a rim of the inner reactor tube, the rim surrounding the inlet opening. Said surface area may act as a resting surface for supporting a top cover assembly, for example.

Preferably, the resting surface of the inner reactor tube may be arranged essentially aligned or even flush with a resting surface of the outer reactor tube. The resting surface of the outer reactor tube may particularly be arranged at the immediate vicinity of the resting surface of the inner reactor tube, thus forming a continuous composite resting surface.

For securing the inner reactor tube to the outer reactor tube, the reactor assembly may further comprise a top cover assembly resting on both the inner reactor tube and the outer reactor tube.

Therefore, the inner reactor tube may be prevented from falling out of the outer reactor tube during operation.

A contamination of a reactor channel or an oxidation chamber within the inner reactor tube in which the oxidation of the sample takes place, may lead to an inaccurate measurement. This may, however, be avoided by providing the top cover assembly with a seal for sealingly engaging at least the inner reactor tube, preferably both the inner reactor tube and the outer reactor tube.

The seal may for example be a sealing ring adapted to be compressed between the outer reactor outer reactor tube and the remainder of the top cover assembly.

Particularly, when working at high temperatures, the top cover assembly may deform, which in turn may hamper the sealing performance of the seal. Hence, it is particularly advantageous if the top cover assembly comprises a unitary flange adapted to be pressed against both the inner and outer reactor tube, the unitary flange being formed of a material having high thermal stability, especially in terms of its thermal expansion. The flange may preferably be formed from a glass-ceramic material, having both the hermetic sealing properties of glass as well as the special properties of ceramics, particularly with respect to high temperature stability against thermal expansion. In this case, an additional sealing ring does not need to be provided between the flange and the resting surface of the inner and/or outer reactor tube.

The flange may comprise a lead through opening penetrating the flange along the longitudinal axis from an end of the flange facing away from the resting surfaces of the inner and outer reactor tube to an end of the flange facing the resting surfaces allowing the insertion of a dispensing needle into a reactor channel of the inner reactor tube.

To seal the lead through opening, the top cover assembly may further comprise a sealing cap adapted to be mounted to the end of the flange facing away from the resting surfaces, for example by means of a threaded joint. The sealing cap may comprise a membrane, particularly a septum, hermetically sealing the lead through opening at the end facing away from the resting surfaces and being penetrable by the dispensing needle.

According to a further advantageous embodiment, the inner reactor tube may at one end protrude from the outer reactor tube along the longitudinal axis. Particularly, the second end of the inner reactor tube comprising the outlet opening may protrude from the outer reactor tube.

In a further advantageous embodiment, a bottom cover assembly may be provided, wherein the bottom cover assembly receives the inner reactor tube, particularly the second end of the inner reactor tube, and sealingly engages the outer reactor tube. Consequently, the oxidised product is prevented from leaking into the surroundings by the seal between the outer reactor tube and the bottom cover assembly.

As a seal is already provided between the outer reactor tube and the bottom cover assembly, a second seal between the inner reactor tube and the bottom cover assembly is not necessary and may thus be omitted.

The bottom cover assembly may comprise a receptacle having a first radially inward protruding step, in which a sealing ring is provided for sealingly engaging the outer reactor tube. A second step may be formed for abutting the outer reactor tube in a direction essentially parallel to the longitudinal axis. The second step may be arranged adjacent to the first step along the longitudinal axis and may extend further radially inwards than the first step.

To support the inner reactor tube, a radially inward protruding step may be provided. If the bottom cover assembly comprises the first and second step, said step for abutting the inner reactor tube may form a third step extending further radially inwards than the first and second step.

In order to direct the outflowing product from the outlet opening to a connection port for connecting a tube leading to a detector, the bottom cover assembly may comprise a funnel tapering in a direction away from the reactor tubes. The funnel may particularly extend from the step for abutting the inner reactor tube along the longitudinal axis away from the inner reactor tube.

Particularly at the interface between the inner reactor tube and the bottom cover assembly, there is a risk of accumulating solid particles. This may be prevented by having the funnel diameter being larger than an inner diameter of the inner reactor tube. Consequently, no solid components of the sample, particularly debris such as soot, may accumulate at the step for abutting the inner reactor tube.

The funnel may comprise a polished surface, prompting the solid particles to glide down the funnel towards the exit instead of being retained on the funnel wall.

For collecting the solid particles, a collection receptacle may be arranged underneath the funnel. The collection receptacle may be particularly aligned with the exit of the funnel, so that the solid particles simply drop into the collection receptacle. The collection receptacle may preferably be a separate part to the funnel, so that it may be removed and emptied as well as easily replaced without having to dismount the funnel from the reactor tubes.

The collection receptacle may, for example, be placed in a further component of the bottom cover assembly, the component being separate from the funnel and detachably mounted to the funnel. The component may be a plastic part adapted to be screwed onto the component comprising the funnel and may further comprise the connection port.

According to a further advantageous embodiment, the reactor assembly may comprise at least two inner reactor tubes configured to be interchangeably inserted into the outer reactor tube. Consequently, a kit may be provided, the kit comprising an outer reactor tube and at least two inner reactor tubes, which are configured to be interchangeably inserted into the outer reactor tube. A quick exchange between inner reactor tubes is possible, further reducing the down time of the analyser.

Usually, a high temperature for oxidation of the sample is needed, particularly for high temperature oxidation methods, like combustion or catalytic oxidation. In such a case, the reactor assembly may be further improved when the inner reactor tube is fittingly inserted into the outer reactor tube. An outer surface of the inner reactor tube thus comes into direct contact with an inner surface of the outer reactor tube establishing a direct heat transfer between the outer and the inner reactor tubes.

In order to further improve the heat transfer between the reactor tubes, the outer reactor tube and the inner reactor tube may be formed from the same material, such as ceramic material. Ceramic materials work particularly well for thermal insulators, which may lead to a low loss of heat within the reactor channel of the inner reactor tube. Hence, the energy required to keep the reactor channel at a certain predetermined temperature may be reduced.

The invention further relates to an industrial water analysis device, particularly for total organic carbon analysis using high temperature oxidation methods such as thermal combustion, the industrial water analysis device comprising a housing and at least one reactor assembly according to any one of the above-mentioned embodiments. The outer reactor tube may be fixedly mounted to the housing and the inner reactor tube may be removably inserted into the outer reactor tube.

The industrial water analysis device may further be adapted to analyse the total nitrogen bound (TN_{b}), total carbon (TC), which represents the sum of the total organic carbon and the total inorganic carbon, and/or the chemical oxygen demand (COD). The measurement for TOC may be in conformity with DIN EN 1484 and the measurement for TN_{b} may be in conformity with DIN EN 12260.

Particularly, when using a thermal combustion method for oxidising the carbon of the sample, a high temperature of about 1200° C. is required to ensure the oxidisation of the entire carbon in the sample to carbon dioxide. For this, the housing may be a heating chamber, in which a heating element, such as a heating coil, is wrapped at least in sections around the outer reactor tube. By energizing the heating unit, the outer reactor tube is heated, which eventually results into heating of the inner reactor tube and the reactor channel.

In order to prevent the reactor assembly from shifting within the housing, bearings may be provided for securing the outer reactor tube within the housing. As the inner reactor tube is held in the outer reactor tube, the bearings are not directly engaged to the inner reactor tube, allowing an easy removal of the inner reactor tube without having to dismantle the housing and/or the bearings.

To further improve the accessibility of the inner reactor tube, the housing may comprise an upper wall having a tube access opening from which the inner reactor tube may be removed from or inserted into the outer reactor tube. To do this, the tube access opening may particularly be arranged coaxially with the inner reactor tube.

At least the top cover assembly may be at least partially arranged outside the housing, so that the top cover assembly is easily accessible and in the case of the housing being a heating chamber, is not directly heated. Therefore, the temperature at the top cover assembly is lower than within the heating chamber, so that the sealing properties do not decrease due to high heat. Furthermore, this results in a broader choice of material for the top cover assembly, which allows cheaper and easily available materials to be used for forming the components of the top cover assembly.

Preferably, the inner reactor tube may protrude from the housing through the tube access opening, such that the first end of the inner reactor tube may be arranged outside the housing.

Thus, the inner reactor tube may be easily accessed for removing the inner reactor tube from the outer reactor tube without having to dismantle the housing.

Additionally or alternatively, the housing may comprise a lower wall being arranged opposite the upper wall along the longitudinal axis, the lower wall having an opening for accessing the reactor assembly. Preferably, the inner reactor tube and/or the outer reactor tube may at least partially protrude from the housing through the opening at the lower wall.

The bottom cover assembly may preferably be mounted to the inner and/or outer reactor tube outside the housing, so that the bottom cover assembly may be easily mounted or detached to the reactor tubes without having to open the housing. This also allows for a quick replacement of a single component of the bottom cover assembly or even the entire bottom cover assembly.

Furthermore, the choice of the type of material for the components of the bottom cover assembly is expanded, as the material is not directly subjected to the conditions within the housing, such as high temperatures.

The invention further relates to an inner reactor tube for a reactor assembly according to any one of the above embodiments.

Optionally, a filling material may be provided within the reactor channel. In case catalytic oxidation is desired, the filling material may be a catalyst. However, catalytic oxidation may result in frequent exchanges as the catalyst may become deactivated and needs to be replaced. Therefore, an analysis method using thermal combustion at high temperatures without the need of a catalyst is preferred. In this case, the filling material may particularly comprise ceramic material having good heat conducting properties for heating the sample within the reactor channel. The filling material may for example be provided in a plurality of spheres that are inserted into the reactor channel. The spherical form of the filling material is particularly advantageous, as the surface area for contacting the sample is further increased.

In order to prevent the filling material from falling out of the reactor channel from the outlet opening, the reactor channel may taper towards the outlet opening. Hence, the packing density towards the outlet opening may be increased, such that the filling material is jammed. However, such a configuration requires a specific geometric shape of the reactor channel, which results in high manufacturing efforts and costs.

Therefore, according to a more preferable embodiment, a gas permeable wall may be provided, the gas permeable wall being mounted within the inner reactor tube for retaining the filling material within the reactor channel. Hence, the inner reactor tube may comprise a regular configuration, particularly shape, and the filling material is still prevented from dropping out of the outlet opening.

The gas permeable wall may extend perpendicular to the longitudinal axis and divide the reactor channel into two sections, one section extending from the inlet opening to the gas permeable wall and a further section extending from the gas permeable wall to the outlet opening. In other words, the gas permeable wall may be arranged within the inner reactor tube distanced from both the inlet opening and the outlet opening. Therefore, the filling material may be held at a specific depth in the reactor channel. The heating elements may be arranged at said height on the outer reactor tube, so that the reactor tube, particularly the filling material reaches its highest temperature at that section. The provision of excessive amounts of filling material for the filling material to tower up to reach said height, is prevented by arranging the gas permeable wall at a predetermined depth within the inner reactor tube. Preferably, the gas permeable wall may be arranged at a bottom quarter of the inner reactor tube proximal to the outlet opening.

More specifically, the gas permeable wall may be arranged at an area between the bottom quarter of the inner reactor tube and a bottom sixth, particularly a bottom fifth, of the inner reactor tube. In other words, the length of the inner reactor tube from the outlet opening to the gas permeable wall should be at least more than 16% or 20% of the inner reactor tube's entire length, respectively, but not more than 25%.

To secure the gas permeable wall within the inner reactor tube, the gas permeable wall may comprise a fixation extension extending from the gas permeable wall along the longitudinal axis. The fixation extension increases the surface area for engaging the inner surface of the inner reactor tube. The fixation extension may, for example, be a tube section having an outer diameter smaller than the inner diameter of the inner reactor tube, so that the tube section may be inserted into the reactor channel and be fixed to the inner reactor tube. The tube section may, for example, adhere to the inner reactor tube, wherein an outer surface of the tube section engages the inner surface of the inner reactor tube.

The gas permeable wall may be formed as a membrane preventing any solid matter to pass through the wall. However, this may lead to an accumulation of solid material from the sample, which in turn may lead to a blockage, and thus require frequent exchange of inner reactor tubes.

Therefore, the gas permeable wall may be a sieve having multiple orifices through which gas but also smaller solid materials may pass. The diameter of the orifices may particularly be smaller than the light width of the filling material, thus preventing the filling material from passing through the sieve.

According to a further advantageous embodiment, the inner reactor tube may comprise at least one tool engagement feature, the at least one tool engagement feature being accessible from outside the reactor assembly, particularly outside the housing of the industrial water analysis device. Hence, the inner reactor tube may be gripped by a tool via the tool engagement feature allowing a safe removal and/or insertion of the inner reactor tube even at high heat.

The tool engagement feature may be formed as a radial notch at the proximity of the inlet opening, such that the inner reactor tube may be engaged by a tool, such as a pair of tongs. Preferably, the notch may be formed on an inner surface of the inner reactor tube, such that the tool engagement feature is easily accessible through the tube access opening of the housing, when the inner reactor tube is inserted into the outer reactor tube.

Alternative to the notch, the tool engagement feature may be formed as a hole radially penetrating a wall of the inner reactor tube.

Hereinafter, exemplary embodiments of the invention are described with reference to the drawings. The embodiments shown and described are for explanatory purposes only. The combination of features shown in the embodiments may be changed according to the foregoing description. For example, a feature which is not shown in an embodiment but described above may be added if the technical effect associated with this feature is beneficial for a particular application and vice versa (a feature shown as part of an embodiment may be omitted as described above, if the technical effect associated with this feature is not needed in a particular application).

In the drawings, elements that correspond to each other with respect to function and/or structure have been provided with the same reference numeral.

In the drawings,

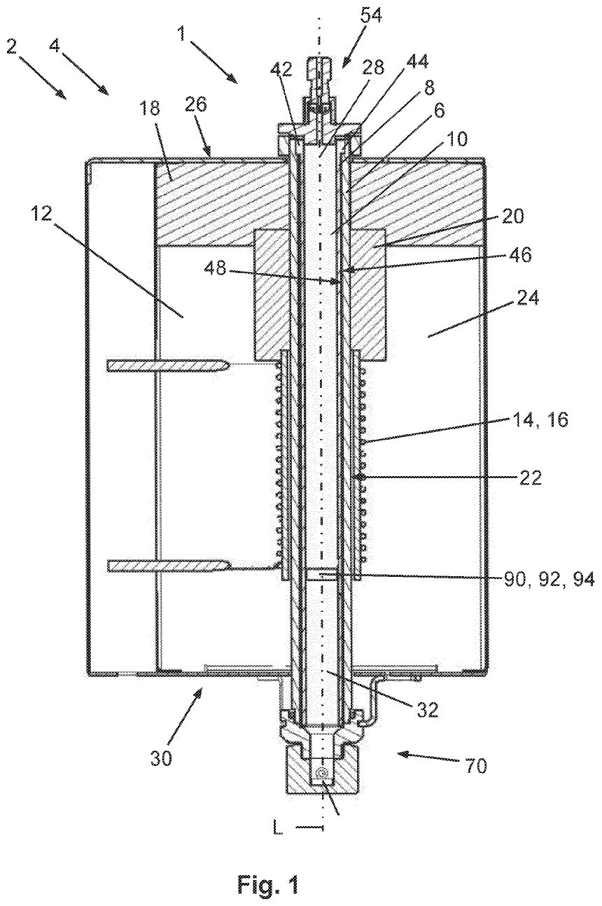

FIG. 1 shows a schematic rendition of a sectional view of an exemplary embodiment of an inventive total organic content analyser;

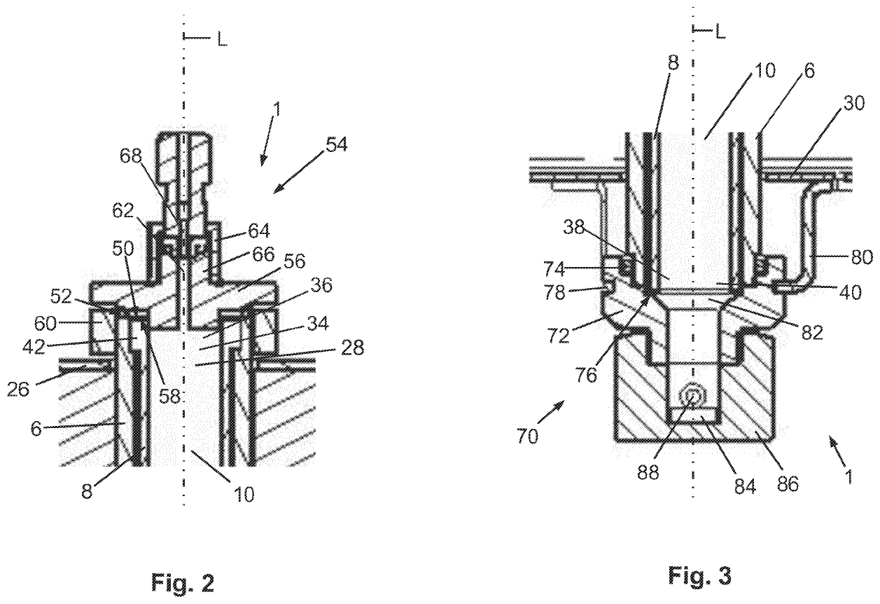

FIG. 2 shows an enlarged partial view of the sectional view from FIG. 1;

FIG. 3 shows a further enlarged partial view of the sectional view from FIG. 1;

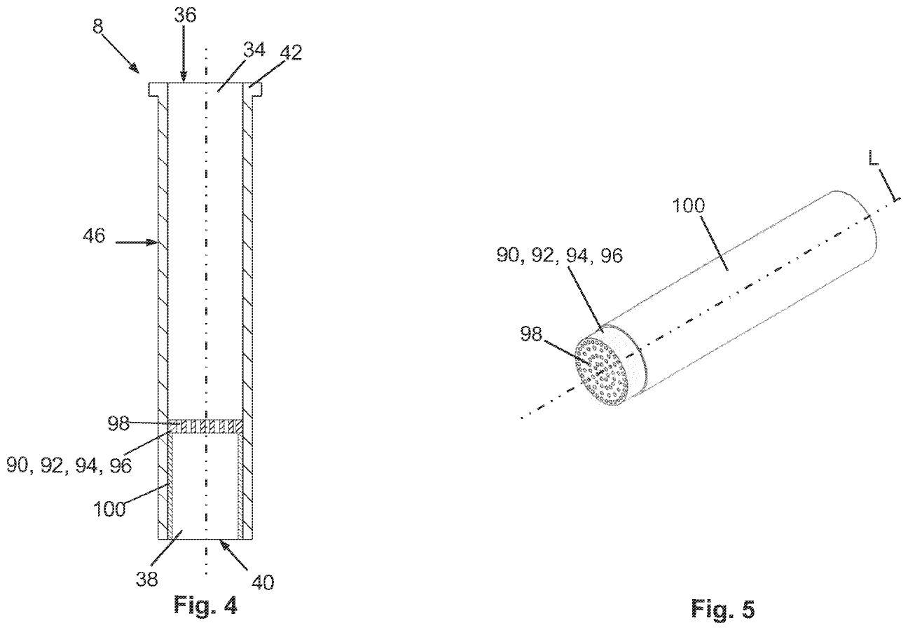

FIG. 4 shows a schematic rendition of a sectional view of an inner reactor tube according to one possible embodiment; and

FIG. 5 shows a schematic rendition of a perspective view of a gas permeable wall shown in FIG. 4.

In the following, the structure of a possible embodiment of a reactor assembly 1 and an industrial water analysis device 2 according to the present invention is explained with reference to the exemplary embodiment shown in FIG. 1.

FIG. 1 shows a sectional view of the industrial water analysis device 2 comprising a housing 4 and an exemplary embodiment of the inventive reactor assembly 1.

Industrial water analysis devices 2 are configured for industrial use, i.e. for use 24/7 to continuously monitor specific content in water, such as TOC or TN_{b} (total nitrogen bound). For this, the industrial water analysis device 2 may be installed on-line or even in-line and may be configured to automatically draw and analyse water samples according to a predetermined schedule. This distinguishes the industrial water analysis device from a water analysis device for laboratory use, which is employed only sporadically.

The industrial water analysis device 2 may further be adapted to analyse the total nitrogen bound (TN_{b}), total carbon (TC), which represents the sum of the total organic carbon and the total inorganic carbon, and/or the chemical oxygen demand (COD). The measurement for TOC may be in conformity with DIN EN 1484 and the measurement for TN_{b} may be in conformity with DIN EN 12260.

The reactor assembly 1 comprises an outer reactor tube 6 and an inner reactor tube 8 being removably inserted into the outer reactor tube 6.

Hence, a single reactor tube is replaced by a combination of an outer reactor tube 6 and an inner reactor tube 8. The outer reactor tube 6 may be fixed to the housing 4 in the same manner a single reactor tube would be fixed in a common total organic content analyser. However, since the inner reactor tube 8 is removably inserted into the outer reactor tube 6, the inner reactor tube 8 may be easily exchanged without having to dismount the outer reactor tube 6 from the housing 4. This means that the downtime during maintenance may be significantly reduced.

During total organic carbon analysis, a sample is introduced into a reactor channel 10 of the inner reactor tube 8, in which the carbon is oxidised to form carbon dioxide. This oxidation step may preferably be performed at high temperatures, such as about 600° C. for a catalytic oxidation or about 1200° C. for a thermal combustion without the presence of a catalyst. Therefore, the housing 4 may constitute a heating chamber 12 having a heating unit 14 to heat up the reactor assembly 1. The heating unit 14 may, for example, be a heating coil 16, being wrapped around the outer reactor tube 6.

To stabilise the reactor assembly 1 within the housing 4, bearings 18, 20 may be provided. The bearings 18, 20 abut an outer surface 22 of the outer reactor tube 6. Therefore, only the outer reactor tube 6 is held directly by the bearings 18, 20 and the inner reactor tube 8 may be moved independently out of and/or into the outer reactor tube 6 with respect to the bearings 18, 20. The bearings may preferably be arranged within the housing 4 and may prevent movement of the reactor assembly 1 at least in a radial direction.

As can be seen in the embodiment shown in FIG. 1, the reactor assembly 1, particularly the inner reactor tube 8 and the outer reactor tube 6, may extend along a longitudinal axis L.

The housing 4 may enclose a volume 24 at least partially receiving the reactor assembly 1. An upper wall 26 extending essentially perpendicular to the longitudinal axis L may limit the volume 24 in a direction essentially parallel to the longitudinal axis L. However, for allowing access at least to the inner reactor tube 8 from outside the housing 4, the upper wall 26 may comprise an access opening 28.

Correspondingly, a lower wall 30 may be provided, the lower wall 30 being arranged opposite the upper wall 26 along the longitudinal axis L. The lower wall 30 may also comprise an opening 32 through which the reactor assembly 1 may at least partially protrude from the housing 4.

In this exemplary embodiment, the inner reactor tube 8 comprises a first end 34 having an inlet opening 36 and a second end 38 having an outlet opening 40. The second end 38 is arranged opposite the first end 34 along the longitudinal axis L. The reactor channel 10 extends essentially parallel to the longitudinal axis L from the inlet opening 36 to the outlet opening 40.

For the sake of clear presentation, the first end 34 of the inner reactor tube 8 is shown in an enhanced view in FIG. 2.

To prevent the inner reactor tube 8 from slipping through the outer reactor tube 6, the inner reactor tube 8 may comprise a radially outwards protruding collar 42 at the first end 34. The collar 42 may be received in a radial recess 44 of the outer reactor tube 6, such that the inner reactor tube 8 at least partially rests on the outer reactor tube 6.

Particularly, the collar 42 may be fittingly received in the recess 44 at least in the radial direction. Therefore, the inner reactor tube 8 may be centred with respect to the outer reactor tube 6, such that the outer and inner reactor tubes 6, 8 are arranged coaxially with one another. This is especially advantageous, as a uniform heat transfer between outer and inner reactor tubes 6, 8 is ensured. To further enhance the heat transfer between outer and inner reactor tubes 6, 8 the inner reactor tube 8 may be fittingly inserted into the outer reactor tube 6, so that an outer surface 46 of the inner reactor tube 8 is in direct contact with an inner surface 48 of the outer reactor tube 6.

The inner reactor tube 8 may comprise a resting surface 50 facing away from the second end 38, whereby the surface area of the resting surface 50 is increased by the provision of the collar 42 at the first end 34. Said resting surface 50 may be essentially aligned or even flush with a resting surface of the outer reactor tube 6 and thus form a composite surface area.

Consequently, it is easier to seal both the inner reactor tube 8 and the outer reactor tube 6 with a single sealing unit, such as a sealing ring 52.

Preferably, the first end 34 of the inner reactor tube 8 may protrude from the housing 4 through the access opening 28, so that the inner reactor tube 8 is easily accessible from outside the housing 4. To further facilitate the removal and/or insertion of the inner reactor tube 8, the inner reactor tube 8 may comprise tool engagement features (not shown) configured to engage a tool, such as a pair of tongs.

In order to secure the inner reactor tube 8 to the outer reactor tube 6, the reactor assembly 1 may further comprise a top cover assembly 54 resting on both the inner reactor tube 8 and the outer reactor tube 6. The top cover assembly 54 may comprise a unitary flange 56 being adapted to rest on the resting surfaces 50. For this, the flange 56 may comprise an abutment surface 58 adapted to at least partially rest on the resting surface 50, whereby the sealing ring 52 may be compressed radially between the abutment surface 58 on one side and an extension 60 of the outer reactor tube 6 extending along the longitudinal axis L from the resting surface 50 of the outer reactor tube 6.

In a particularly advantageous embodiment, the flange 56 may be formed of a glass-ceramic material, having both the hermetic sealing properties of glass as well as the special properties of ceramics, particularly with respect to high temperature stability against thermal expansion.

For allowing the insertion of the sample without having to remove the flange 56, the flange 56 may comprise a lead through opening 62 penetrating the flange 56 along the longitudinal axis L. A sealing cap 64 may be mounted to an end 66 of the flange 56 facing away from the reactor tubes 6, 8. The sealing cap 64 may comprise a seal that can be penetrated by a dispensing needle 68. Preferably, the diameter of the lead through opening 62 may be larger than the diameter of the dispensing needle 68, so that the dispensing needle 68 does not come into physical contact with the flange 56. Therefore, excessive heating up of the sample inside the dispensing needle and possibly already evaporating a portion of the sample in the dispensing needle may be prevented.

The top cover assembly 54 may preferably be arranged outside the housing 4. Therefore, the top cover assembly 54 may easily be detached from the outer and inner reactor tubes 6, 8 without having to open the housing 4. Consequently, the heating unit 14 does not need to be switched off for mounting or dismounting the top cover assembly 54.

As can be seen in FIGS. 1 and 3, the second end 38 of the inner reactor tube 8 may protrude from the housing 4 through the opening 32 of the lower wall 30. Correspondingly, the outer reactor tube 6 may also protrude from the housing 4 through the opening 32. However, according to a preferred embodiment, the second end 38 of the inner reactor tube 8 may extend further along the longitudinal axis L than the outer reactor tube 6, such that the second end 38 projects out of the outer reactor tube 6.

A bottom cover assembly 70 may be provided, the bottom cover assembly 70 being mounted to the outer reactor tube 6 outside the housing 4. The outer reactor tube 6 may be pressed into an interface part 72 of the bottom cover assembly 70, wherein a sealing ring 74 is radially compressed between the interface part 72 and the outer surface 22 of the outer reactor tube 6.

The inner reactor tube 8, however, may simply abut a radially inward protruding step 76 of the interface part 72 along the longitudinal axis L. Preferably, a seal between inner reactor tube 8 and bottom cover assembly 70, particularly the interface part 72 is not provided. Therefore, the inner reactor tube 8 may easily be removed from or inserted into the outer reactor tube 6 without having to disassemble the bottom cover assembly 70.

On the outer side, the interface part 72 may comprise a circumferentially extending notch 78 in which clamps 80 may be inserted for fixing the bottom cover assembly 70, particularly the interface part 72 to the lower wall 30 of the housing 4.

The interface part 72 may preferably be formed from a material with high thermal stability, particularly in terms of its thermal expansion. For example, the interface part 72 may comprise or consist of a ceramic material. Thus, even when the reactor tubes 6, 8 are heated, the interface part 72 does not expand in such a manner that the sealing between the outer reactor tube 6 and the interface part 72 degenerates.

In order to direct the product exiting the inner reactor tube 8 from the outlet opening 40 and preventing an accumulation of solid materials at the interface part 72, the interface part 72 may comprise a funnel 82 tapering in a direction essentially parallel to the longitudinal axis L away from the reactor tubes 6, 8.

At the interface between interface part 72 and inner reactor tube 8, the funnel diameter may at least be larger than an inner diameter of the inner reactor tube 8. Hence, the solid material directly falls into the funnel 82 instead of accumulating at the step 76. Preferably, at least the surface of the funnel may be polished so that the material slides off the surface instead of being retained on the surface. In this case, the maintenance required for cleaning or possibly exchanging the interface part 72 may be significantly reduced.

The bottom cover assembly 70 may further comprise a collection receptacle 84 arranged underneath the funnel 82 along the longitudinal axis L, in order to collect the solid material exiting the inner reactor tube 8. The collection receptacle 84 may be seated in a separate component 86 of the bottom cover assembly 70, the component 86 being detachable from the interface part 72.

The separate component 86 may be provided with a port 88 for connecting the reactor assembly 1 to a detector (not shown). The carbon dioxide may be conducted through the port 88 to the detector, which is configured to measure the amount of carbon dioxide.

Preferably, the separate component 86 may be mounted to the interface part 72 via a threaded connection. Hence, the separate component 86 may be easily removed, since the connection of the bottom cover assembly 70 to the housing 4 and the outer reactor tube 6 is formed by the interface part 72.

Since the separate component 86 is not in direct contact with the reactor tubes 6, 8, the separate component 86 is not subjected to as high heats as the interface part 72. Hence, the choice of material for the separate component is widened allowing the separate component 86, for example, to comprise or consist of a plastic material, which is cheaper and easily available.

As can be seen in FIG. 1, an insert 90 may be provided, the insert 90 being mounted within the reactor channel 10 forming a bottom wall 92 extending perpendicular to the longitudinal axis L. The bottom wall 92 may be a gas permeable wall 94 such that the oxidised carbon dioxide may pass through the gas permeable wall 94 towards the outlet opening 40. Larger solid particles may thus be prevented from falling out of the inner reactor tube 8 and eventually blocking the port 88.

The gas permeable wall 94 may be fixed within the inner reactor tube 8 between a bottom quarter and a bottom sixth of the entire length of the inner reactor tube 8 along the longitudinal axis L proximal to the second end 38. In such a way, the gas permeable wall 94 may be aligned with an end of the heating unit 14, when mounted in the industrial water analysis device as is depicted in FIG. 1. Consequently, a section of the inner reactor tube 8 extending from the gas permeable wall 94 towards the first end 34 may be surrounded by the heating unit 14 such that the highest temperature in the reactor channel 10 is reached at said point. Therefore, the oxidation step may already be completed before the sample reaches the gas permeable wall 94.

The gas permeable wall 94 may be fixed to the inner reactor tube 8 via form fit, press fit and/or adherence.

With reference to FIG. 4, a second exemplary embodiment of the inner reactor tube 8 is depicted, in which a different insert 90 is mounted within the inner reactor tube 8. A schematic perspective view of the insert 90 of the second embodiment is shown in FIG. 5. For the sake of brevity, only the differences to the first embodiment shown in FIG. 1 are discussed hereinafter.

The gas permeable wall 94 is formed as a sieve 96 comprising multiple orifices 98 through which not only gases but also smaller solid particles may pass, allowing for solid particles to be removed from the inner reactor tube 8. Said particles may be collected in the collection receptacle 84 and subsequently removed from the system, without having to remove the inner reactor tube 8.

Furthermore, a fixation extension 100 extending along the longitudinal axis L from the gas permeable wall 94 towards the second end 38 may be provided. The fixation extension 100 may be a tube having a smaller diameter than the inner diameter of the inner reactor tube 8, so that the fixation extension may be inserted into the reactor channel 10. Preferably, the fixation extension 100 may be fittingly inserted into the reactor channel 10 resulting in an abutment of an outer surface of the fixation extension with an inner surface of the inner reactor tube 8. Thus, the surface area available for fixing the gas permeable wall 94 to the inner reactor tube 8 may be significantly increased.

The fixation extension may preferably be aligned with the outlet opening 40 at an end facing away from the gas permeable wall 94.

A filling material (not shown) such as ceramic spheres, may be inserted into the reactor channel through the inlet opening 36, whereby the gas permeable wall 94 may prevent the filling material from falling out of the outlet opening 40.

### REFERENCE NUMERALS

1 reactor assembly
2 industrial water analysis device
4 housing
6 outer reactor tube
8 inner reactor tube
10 reactor channel
12 heating chamber
14 heating unit
16 heating coil
18 bearing
20 bearing
22 outer surface
24 volume
26 upper wall
28 access opening
30 lower wall
32 opening
34 first end
36 inlet opening
38 second end
40 outlet opening
42 collar
44 recess
46 outer surface
48 inner surface
50 resting surface
52 sealing ring
54 top cover assembly
56 flange
58 abutment surface
60 extension
62 lead through opening
64 sealing cap
66 end of flange
68 dispensing needle
70 bottom cover assembly
72 interface part
74 sealing ring
76 step
78 notch
80 clamp
82 funnel
84 collection receptacle
86 component
88 port
90 insert
92 bottom wall
94 gas permeable wall
96 sieve
98 orifice
100 fixation extension
L longitudinal axis

1-23. (canceled)

24. Reactor assembly for an industrial water analysis device, particularly a total organic carbon analyser using a high temperature oxidation method, the reactor assembly comprising an outer reactor tube and an inner reactor tube being removably inserted into the outer reactor tube.

25. Reactor assembly according to claim 24, wherein the inner reactor tube at least partially rests on the outer reactor tube.

26. Reactor assembly according to claim 24, wherein the inner reactor tube comprises a radially protruding collar received in a recess of the outer reactor tube.

27. Reactor assembly according to claim 24, wherein the inner reactor tube comprises a resting surface, the resting surface being aligned with a surrounding resting surface of the outer reactor tube.

28. Reactor assembly according to claim 24, wherein the reactor assembly further comprises a top cover assembly resting on both the inner reactor tube and the outer reactor tube.

29. Reactor assembly according to claim 24, wherein the inner reactor tube at least at one end protrudes from the outer reactor tube.

30. Reactor assembly according to claim 24, wherein a bottom cover assembly is provided receiving the inner reactor tube and sealingly engaging the outer reactor tube.

31. Reactor assembly according to claim 30, wherein the bottom cover assembly comprises a funnel having a funnel diameter larger than an inner diameter of the inner reactor tube.

32. Reactor assembly according to claim 30, wherein the bottom cover assembly comprises a collection receptacle arranged underneath an outlet opening of the inner reactor tube.

33. Reactor assembly according to claim 31, wherein the collection receptacle is arranged underneath the funnel.

34. Reactor assembly according to claim 24, wherein the inner reactor tube and the outer reactor tube are formed of the same material.

35. Industrial water analysis device, particularly for total organic carbon analysis using a high temperature oxidation method, the industrial water analysis device comprising a housing and a reactor assembly according to claim 24, wherein the outer reactor tube is fixedly mounted to the housing.

36. Industrial water analysis device according to claim 35, wherein the housing comprises an upper wall having an access opening and wherein at least the inner reactor tube is accessible through the access opening from outside the housing.

37. Industrial water analysis device according to claim 35, wherein a heating unit is provided within the housing for heating the reactor assembly.

38. Industrial water analysis device according to claim 35, wherein at least the inner reactor tube protrudes from the housing from two opposing sides.

39. Industrial water analysis device according to claim 35, wherein at least one of the top cover assembly and bottom cover assembly is provided and wherein the top cover assembly and bottom cover assembly, respectively, is arranged outside the housing.

40. Inner reactor tube for a reactor assembly according to claim 24, wherein a gas permeable wall is provided, the gas permeable wall being mounted within the inner reactor tube.

41. Inner reactor tube according to claim 40, wherein the gas permeable wall is arranged in a bottom quarter of the inner reactor tube.

42. Inner reactor tube according to claim 41, wherein the gas permeable wall is distanced from the outlet opening of the inner reactor tube.

43. Inner reactor tube according to claim 40, wherein the gas permeable wall is formed as a sieve.

44. Inner reactor tube according to claim 40, wherein a fixation extension extends from the gas permeable wall.

45. Inner reactor tube according to claim 41, wherein the inner reactor tube comprises at least one tool engagement feature.

46. Inner reactor tube according to claim 40, wherein the inner reactor tube further comprises a filling material arranged between an inlet opening and the gas permeable wall.
